(19) United States
(12) Patent Application Publication
(10) Pub. No.: US 2024/0180793 A1
(43) Pub. Date: Jun. 6, 2024

(54) CORE STICK-TYPE OIL DISPERSION SOLID COSMETIC COMPOSITION HAVING IMPROVED SPREADABILITY

(71) Applicant: COSMAX, INC., Gyeonggi-do (KR)

(72) Inventors: Ji Young KIM, Gyeonggi-do (KR); Hyeon Joo LIM, Gyeonggi-do (KR); Jin Goo JI, Gyeonggi-do (KR); Myeong Sam PARK, Seoul (KR)

(21) Appl. No.: 17/798,630

(22) PCT Filed: Nov. 15, 2021
(86) PCT No.: PCT/KR2021/016646

§ 371 Date: Aug. 10, 2022

(30) Foreign Application Priority Data
Sep. 30, 2021 (KR) 10-2021-0129999

(51) Int. Cl. A61K 8/02; A61K 8/25; A61K 8/34; A61K 8/39; A61K 8/49; A61K 8/58; A61K 8/891; A61K 8/92; A61Q 19/02

(52) U.S. Cl. CPC A61K 8/0229; A61K 8/25; A61K 8/342; A61K 8/39; A61K 8/4973; A61K 8/585; A61K 8/891; A61K 8/922; A61Q 19/02

(57) ABSTRACT

The present invention relates to a core stick-type oil dispersion solid cosmetic composition with improved spreadability, wherein the core stick-type oil dispersion solid cosmetic composition contains a core part as a base with high hardness and an outer part as a base with low hardness, and thus is highly effective in wrinkle relief and skin whitening while exhibiting smooth spreadability and a formulation that is not prone to softening.

### TECHNICAL FIELD

This application claims priority to Korean Patent Application No. 10-2021-0129999 filed in the Korean Intellectual Property Office on 30 Sep. 2021, the disclosure of which is incorporated herein by reference.

The present disclosure relates to a core stick-type oil-dispersed solid cosmetic composition having improved spreadability.

### BACKGROUND ART

Conventional stick type oil-dispersed cosmetic compositions have been recently receiving wide attention from consumers since the compositions require no separate product, such as a puff, can be easily applied to the skin and thus are convenient to use, have an excellent moisturizing feeling, and are handy to carry. The oil-dispersed cosmetic compositions have been widely used as moisturizers due to the advantage of helping the skin by forming an oil film when applied to the skin in a mixture with a high content of oil.

However, conventional stick-type oil dispersion stick-type cosmetic materials are easily broken by the pressure applied during application if they are in a soft formulation, and thus in order to prevent this, the cosmetic materials are composed to have high hardness and be unbreakable upon application. Such cosmetic materials having high hardness and unbreakability show a deterioration in spreadability and a decrease in the amount of application on the skin upon application. A feeling in use of smooth and soft formulations is favorable, but such features affect the quality of the formulations, so that a selection is needed between the feeling in use and the formulation quality.

Therefore, there is an urgent need to develop an oil dispersion solid cosmetic composition that is improved in spreadability and is not softened.

### DISCLOSURE OF INVENTION

### Technical Problem

The present inventors have made intensive research efforts to develop an oil dispersion solid cosmetic composition having improved spreadability and increased formulation hardness. As a result, the present inventors confirmed that an oil dispersion solid cosmetic composition containing a core part as a base with high hardness and an outer part as a base with low hardness is effective in wrinkle relief and skin whitening while exhibiting smooth spreadability and a formulation that is not prone to softening.

Accordingly, an aspect of the present disclosure is to provide a core stick-type oil dispersion solid cosmetic composition containing a core part and an outer part.

### Solution to Problem

Hereinafter, the present disclosure will be described in more detail.

An aspect of the present disclosure is directed to a core stick-type oil dispersion solid cosmetic composition containing a core part and an outer part.

In the present disclosure, the weight ratio between the core part and the outer part may be 10-70:30-90, 10-60:40-90, 10-50:50-90, 10-40:60-90, 10-30:70-90, 20-70:30-80, 20-60:40-80, 20-50:50-80, 20-40:60-80, or 20-30:70-80, and for example, 25:75, but is not limited thereto.

In the present disclosure, the core part may contain at least one selected from the group consisting of an ester oil, a vegetable oil, and a wax, but is not limited thereto.

In the present disclosure, the ester oil may be at least one selected from the group consisting of polyglyceryl-2 triisostearate, octyldodecanol, cetyl ethylhexanoate, triethylhexanoin, isopropyl myristate, dicaprylyl carbonate, and pentaerythrityl tetraethylhexanoate, but is not limited thereto.

In the present disclosure, the ester oil may be contained, relative to the total cosmetic composition, in a content of 1.0 to 20.0 wt %, 1.0 to 17.0 wt %, 1.0 to 14.0 wt %, 1.0 to 11.0 wt %, 1.0 to 9.0 wt %, 3.0 to 20.0 wt %, 3.0 to 17.0 wt %, 3.0 to 14.0 wt %, 3.0 to 11.0 wt %, 3.0 to 9.0 wt %, 6.0 to 20.0 wt %, 6.0 to 17.0 wt %, 6.0 to 14.0 wt %, 6.0 to 11.0 wt %, or 6.0 to 9.0 wt %, and for example, 8.0 wt %, but is not limited thereto.

In the present disclosure, the vegetable oil may be at least one selected from the group consisting of sunflower seed oil, sweet almond oil, Simmondsia chinensis (Jojoba) seed oil, avocado oil, Macadamia integrifolia seed oil, olive oil, meadowfoam seed oil, grape seed oil, canola oil, Ricinus communis (Castor) seed oil, and corn oil, but is not limited thereto.

In the present disclosure, the vegetable oil may be contained, relative to the total cosmetic composition, in a content of 1.0 to 30.0 wt %, 1.0 to 25.0 wt %, 1.0 to 20.0 wt %, 1.0 to 15.0 wt %, 5.0 to 30.0 wt %, 5.0 to 25.0 wt %, 5.0 to 20.0 wt %, 5.0 to 15.0 wt %, 10.0 to 30.0 wt %, 10.0 to 25.0 wt %, 10.0 to 20.0 wt %, or 10.0 to 15.0 wt %, and for example, 13 wt %, but is not limited thereto.

In the present disclosure, the wax may be at least one selected from the group consisting of candelilla wax, microcrystalline wax, ceresin, polyethylene, paraffin, and ozokerite, but is not limited thereto.

In the present disclosure, the wax may be contained, relative to the total cosmetic composition, in a content of 20.0 to 60.0 wt %, 20.0 to 55.0 wt %, 20.0 to 50.0 wt %, 20.0 to 45.0 wt %, 20.0 to 40.0 wt %, 25.0 to 60.0 wt %, 25.0 to 55.0 wt %, 25.0 to 50.0 wt %, 25.0 to 45.0 wt %, 25.0 to 40.0 wt %, 30.0 to 60.0 wt %, 30.0 to 55.0 wt %, 30.0 to 50.0 wt %, 30.0 to 45.0 wt %, 30.0 to 40.0 wt %, 35.0 to 60.0 wt %, 35.0 to 55.0 wt %, 35.0 to 50.0 wt %, or 35.0 to 45.0 wt %, and for example, 40.0 wt %, but is not limited thereto.

In the present disclosure, the outer part may contain at least one selected from the group consisting of an ester oil, a silicone oil, a silica powder, a wax, a surfactant, and a thickener, but is not limited thereto.

In the present disclosure, the ester oil may be at least one selected from the group consisting of cetyl ethylhexanoate, triethylhexanoin, polyglyceryl-2 triisostearate, octyldodecanol, cetyl ethylhexanoate, triethylhexanoin, isopropyl myristate, dicaprylyl carbonate, and pentaerythrityl tetraethylhexanoate, but is not limited thereto.

In the present disclosure, the ester oil may be contained, relative to the total cosmetic composition, in a content of 5.0 to 30.0 wt %, 5.0 to 25.0 wt %, 5.0 to 20.0 wt %, 5.0 to 15.0 wt %, 10.0 to 30.0 wt %, 10.0 to 25.0 wt %, 10.0 to 20.0 wt %, or 10.0 to 15.0 wt %, and for example, 15.0 wt %, but is not limited thereto.

In the present disclosure, the silicone oil may be at least one selected from the group consisting of dimethicone, phenyl trimethicone, cyclopentasiloxane, cyclohexasiloxane, caprylyl methicone, PEG-11 methyl ether dimethicone, cetyl dimethicone, and PEG-12 dimethicone, but is not limited thereto.

In the present disclosure, the silicone oil may be contained, relative to the total cosmetic composition, in a content of 1.0 to 20.0 wt %, 1.0 to 18.0 wt %, 1.0 to 16.0 wt %, 1.0 to 14.0 wt %, 1.0 to 12.0 wt %, 5.0 to 20.0 wt %, 5.0 to 18.0 wt %, 5.0 to 16.0 wt %, 5.0 to 14.0 wt %, 5.0 to 12.0 wt %, 8.0 to 20.0 wt %, 8.0 to 18.0 wt %, 8.0 to 16.0 wt %, 8.0 to 14.0 wt %, or 8.0 to 12.0 wt %, and for example, 11.0 wt %, but is not limited thereto.

In the present disclosure, the silica powder may be at least one selected from the group consisting of polymethylsilsesquioxane (PMSQ), vinyl dimethicone/methicone silsesquioxane crosspolymer, silica, and silica silylate, but is not limited thereto.

In the present disclosure, the silica powder may be contained, relative to the total cosmetic composition, in a content of 1.0 to 10.0 wt %, 1.0 to 8.0 wt %, 1.0 to 6.0 wt %, 3.0 to 10.0 wt %, 3.0 to 8.0 wt %, or 3.0 to 6.0 wt %, and for example, 5.0 wt %, but is not limited thereto.

In the present disclosure, the wax may be at least one selected from the group consisting of hydrogenated vegetable oil, polyethylene, paraffin, microcrystalline wax, candelilla wax, ceresin, polyethylene, and ozokerite, but is not limited thereto.

In the present disclosure, the wax may be contained, relative to the total cosmetic composition, in a content of 1.0 to 10.0 wt %, 1.0 to 8.0 wt %, 1.0 to 6.0 wt %, 1.0 to 4.0 wt %, 3.0 to 10.0 wt %, 3.0 to 8.0 wt %, or 3.0 to 6.0 wt %, and for example, 4.0 wt %, but is not limited thereto.

In the present disclosure, the surfactant may be at least one selected from the group consisting of sorbitan isostearate, polyglyceryl-4 isostearate, sorbitan oleate, polyglyceryl-2 triisostearate, polyglyceryl-2 diisostearate, and polyglyceryl-10 laurate, but is not limited thereto.

In the present disclosure, the surfactant may be contained, relative to the total cosmetic composition, in a content of 0.1 to 3.0 wt %, 0.1 to 2.5 wt %, 0.1 to 2.0 wt %, 0.1 to 1.5 wt %, 0.1 to 1.0 wt %, 0.5 to 3.0 wt %, 0.5 to 2.5 wt %, 0.5 to 2.0 wt %, 0.5 to 1.5 wt %, 0.5 to 1.0 wt %, and for example, 1.0 wt %, but is not limited thereto.

In the present disclosure, the thickener may be at least one selected from the group consisting of diisostearyl malate, VP/hexadecene copolymer, silica dimethyl silylate, polybutene, and hydrogenated polyisobutene, but is not limited thereto.

In the present disclosure, the thickener may be contained, relative to the total cosmetic composition, in a content of 1.0 to 10.0 wt %, 1.0 to 8.0 wt %, 1.0 to 5.0 wt %, 2.0 to 10.0 wt %, 2.0 to 8.0 wt %, or 2.0 to 5.0 wt %, and for example, 3.0 wt %, but is not limited thereto.

### Advantageous Effects of Invention

The present disclosure is directed to a core stick-type oil dispersion solid cosmetic composition with improved spreadability, wherein the core stick-type oil dispersion solid cosmetic composition contains a core part as a base with high hardness and an outer part as a base with low hardness, and thus is highly effective in wrinkle relief and skin whitening while exhibiting smooth spreadability and a formulation that is not prone to softening.

### BRIEF DESCRIPTION OF DRAWINGS

FIG. 1 is an image showing the formulation of Example 1 according to one preparative example of the present disclosure.

FIG. 2 is an image showing the spreadability comparison results between Example 1 and Comparative Example 1 according to one preparative example of the present disclosure.

FIG. 3 is an image showing the formulation stability comparison results between Example 1 and Comparative Example 1 according to one preparative example of the present disclosure.

### BEST MODE FOR CARRYING OUT THE INVENTION

A core stick-type oil dispersion solid cosmetic composition containing a core part and an outer part

### MODE FOR CARRYING OUT THE INVENTION

Hereinafter, the present disclosure will be described in more detail by the following examples. However, these exemplary embodiments are used only for illustration, and the scope of the present disclosure is not limited by these exemplary embodiments.

### Preparative Example: Preparation of Example and Comparative Examples

Core stick-type oil dispersion solid cosmetic compositions of Example 1 and Comparative Example 1 were prepared according to the ingredients and contents thereof shown in Table 1 below.

TABLE 1

| | | | | | Comparative |
|---|---|---|---|---|---|
| NO | Classification | | Ingredient | Example 1 | Example 1 |
| 1 | Core part | Ester oil | Polyglyceryl-2 triisostearate | 5.0 | — |
| 2 | | | Octyldodecanol | 3.0 | — |
| 3 | | Vegetable | Sunflower seed oil | 9.0 | — |
| 4 | | oil | Sweet almond oil | 4.0 | — |
| 5 | | Wax | Candelilla wax | 35.0 | — |
| 6 | | | Microcrystalline wax | 5.0 | — |
| 7 | Outer part | Ester oil | Cetyl ethylhexanoate | 10.0 | 15.5 |
| 8 | | | Triethylhexanoin | 5.0 | 5.0 |
| 9 | | Silicon oil | Phenyl trimethicone | 8.0 | 5.0 |
| 10 | | | Dimethicone | 3.0 | 9.0 |
| 11 | | Silica | Polymethylsilsesquioxane | 2.5 | 2.0 |
| 12 | | powder | Silica | 1.5 | 2.3 |
| 13 | | | Silica silylate | 0.5 | 1.2 |
| 14 | | | Vinyl dimethicone/methicone | 0.5 | 1.0 |
| | | | silsesquioxane crosspolymer | | |
| 15 | | Wax | Paraffin | 2.0 | 20.0 |
| 16 | | | Microcrystalline wax | 1.0 | 13.0 |
| 17 | | | Polyethylene | 1.0 | 20.0 |
| 18 | | Surfactant | Sorbitan isostearate | 1.0 | 1.0 |
| 19 | | Thickener | Diisostearyl malate | 2.0 | 3.0 |
| 20 | | | Silica dimethyl silylate | 0.5 | 1.5 |
| 21 | | | VP/hexadecene copolymer | 0.5 | 0.5 |

| | | |
|---|---|---|
| Total (unit: wt %) | 100.0 | 100.0 |

Specifically, the raw materials for an outer part in Table 1 were introduced in the order of the waxes, thickeners, surfactant, ester oils, and silicone oils, and then mixed and dissolved by an Agi-mixer at 25 rpm for 10 minutes while warmed to 80-85° C. After confirming a state of being dissolved, silica powders were slowly added with stirring by the Agi-mixer, followed by further stirring at 25 rpm for 10 minutes. Then, the completely dissolved contents were defoamed.

Specifically, the raw materials for a core part in Table 1 were introduced to a beaker in the order of the waxes, ester oils, and vegetable oils, and then mixed by an Agi-mixer at 25 rpm for 10 minutes while warmed to 80-85° C. Then, the completely dissolved contents were defoamed.

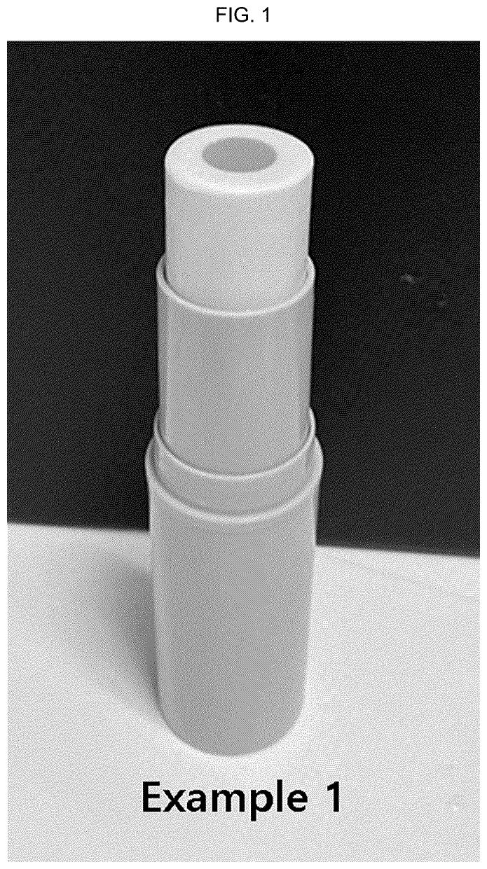

The prepared outer part contents were poured in a core mold at 78 to 82° C. and cooled at −18° C. for 10 minutes, thereby setting an outer part. The prepared core part contents were poured into the outer part set at 78 to 82° C. and cooled at −18° C. for 10 minutes, thereby preparing a core stick-type oil dispersion solid cosmetic composition composed of a core part and an outer part (FIG. 1).

### Test Example 1: Evaluation of Formulation Stability

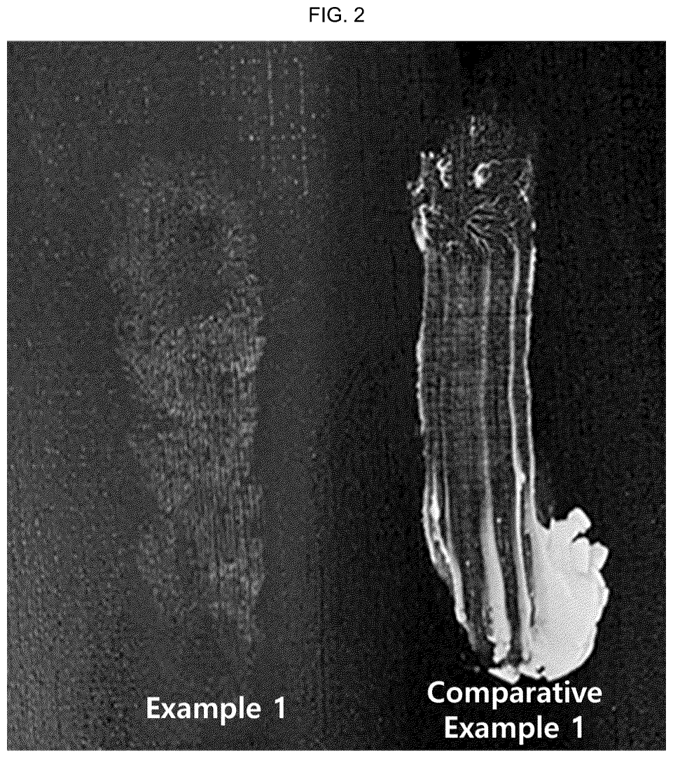

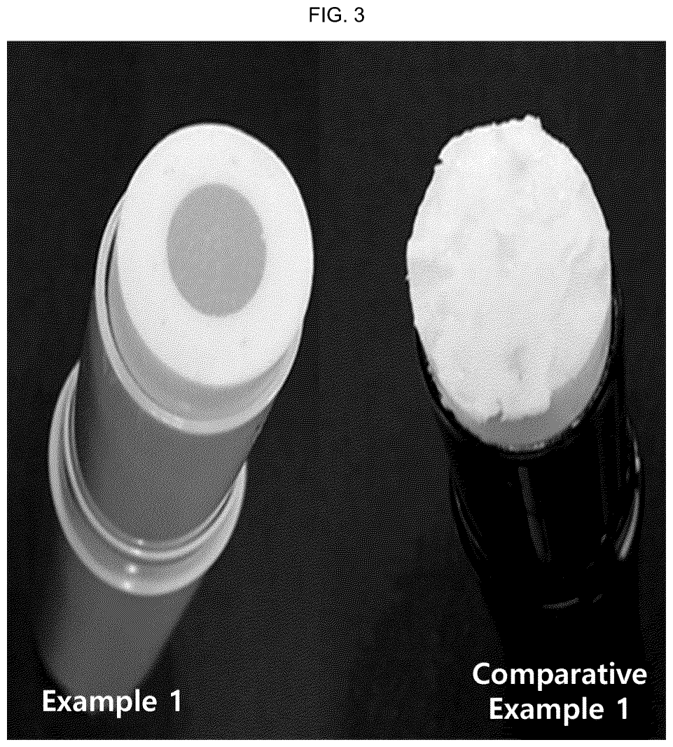

Example 1 and Comparative Example 1 were compared for formulation stability. Each solid cosmetic composition filled to the same height was applied to a black plate at an interval of 5 cm by using the same force. The results of application and the formulations of cosmetic compositions were shown in FIGS. 2 and 3.

As can be confirmed from FIGS. 2 and 3, Example 1 was less softened to exhibit stable spreadability, and the formulation of the cosmetic composition was hardly changed after application. However, Comparative Example 1 was non-uniformly applied, and the formulation of the cosmetic composition was softened after application. The reason why the core stick-type oil dispersion solid cosmetic composition of Example 1 exhibited stable spreadability is that when the contents obtained by mixing 75% of the outer part with a smooth and soft feature and 25% of the core part are applied, the core part structurally and stably maintained the formulation of the cosmetic composition.

### Test Example 2 Evaluation of Whitening Effect

To evaluate the whitening effect of Example 1, the tyrosinase activity inhibitory effect was determined. Mushroom tyrosinase inhibition assay was used, and 10 μl of Example 1 and 20 μl of an aqueous solution of mushroom tyrosinase (1000 units/mL) were added to 170 μl of a reaction mixture obtained by mixing L-tyrosine, 50 mM potassium phosphate buffer (pH 6.5), and distilled water at a ratio of 10:10:9, followed by incubation at 25° C. for 30 minutes. Then, the absorbance was recorded at 490 nm by a microplate reader to determine the amount of dopachrome generated. Arbutin and kojic acid, which are known as tyrosinase activity inhibiting substances, were used as positive controls, and the measurement results are shown in Table 2. The expression of the percentage of tyrosinase activity inhibition is as follows.

Tyrosinase activity inhibitory effect (%)=(1−(A−B)/C)×100 [Equation]
A: Absorbance of sample
B: Absorbance of color control
C: Absorbance of control

TABLE 2

| Sample | | IC50 (unit: μg/ml) |
|---|---|---|
| Example 1 | | 30.5 |
| Control | Arbutin | 630.1 |
| | Kojic acid | 35.3 |

As can be confirmed from Table 2, Example 1 showed a whitening effect through high tyrosinase activity inhibitory ability.

### INDUSTRIAL APPLICABILITY

The present disclosure relates to a core stick-type oil-dispersed solid composition having improved spreadability.

1. A core stick-type oil dispersion solid cosmetic composition comprising a core part and an outer part.

2. The core stick-type oil dispersion solid cosmetic composition of claim 1, wherein the weight ratio between the core part and the outer part is 10-70:30-90.

3. The core stick-type oil dispersion solid cosmetic composition of claim 1, wherein the core part comprises at least one selected from the group consisting of an ester oil, a vegetable oil, and a wax.

4. The core stick-type oil dispersion solid cosmetic composition of claim 3, wherein the ester oil is at least one selected from the group consisting of polyglyceryl-2 triisostearate, octyldodecanol, cetyl ethylhexanoate, triethylhexanoin, isopropyl myristate, dicaprylyl carbonate, and pentaerythrityl tetraethylhexanoate.

5. The core stick-type oil dispersion solid cosmetic composition of claim 3, wherein the ester oil is contained in a content of 1.0 to 20.0 wt % relative to the total cosmetic composition.

6. The core stick-type oil dispersion solid cosmetic composition of claim 3, wherein the vegetable oil is at least one selected from the group consisting of sunflower seed oil, sweet almond oil, Simmondsia chinensis (Jojoba) seed oil, avocado oil, Macadamia integrifolia seed oil, olive oil, meadowfoam seed oil, grape seed oil, canola oil, Ricinus communis (Castor) seed oil, and corn oil.

7. The core stick-type oil dispersion solid cosmetic composition of claim 3, wherein the vegetable oil is contained in a content of 1.0 to 30.0 wt % relative to the total cosmetic composition.

8. The core stick-type oil dispersion solid cosmetic composition of claim 3, wherein the wax is at least one selected from the group consisting of candelilla wax, microcrystalline wax, ceresin, polyethylene, paraffin, and ozokerite.

9. The core stick-type oil dispersion solid cosmetic composition of claim 3, wherein the wax is contained in a content of 20.0 to 60.0 wt % relative to the entire cosmetic composition.

10. The core stick-type oil dispersion solid cosmetic composition of claim 1, wherein the outer part comprises at least one selected from the group consisting of an ester oil, a silicone oil, a silica powder, a wax, a surfactant, and a thickener.

11. The core stick-type oil dispersion solid cosmetic composition of claim 10, wherein the ester oil is at least one selected from the group consisting of cetyl ethylhexanoate, triethylhexanoin, polyglyceryl-2 triisostearate, octyldodecanol, cetyl ethylhexanoate, triethylhexanoin, isopropyl myristate, dicaprylyl carbonate, and pentaerythrityl tetraethylhexanoate.

12. The core stick-type oil dispersion solid cosmetic composition of claim 10, wherein the ester oil is contained in a content of 5.0 to 30.0 wt % relative to the total cosmetic composition.

13. The core stick-type oil dispersion solid cosmetic composition of claim 10, wherein the silicone oil is at least one selected from the group consisting of dimethicone, phenyl trimethicone, cyclopentasiloxane, cyclohexasiloxane, caprylyl methicone, PEG-11 methyl ether dimethicone, cetyl dimethicone, and PEG-12 dimethicone.

14. The core stick-type oil dispersion solid cosmetic composition of claim 10, wherein the silicone oil is contained in a content of 1.0 to 20.0 wt % relative to the total cosmetic composition.

15. The core stick-type oil dispersion solid cosmetic composition of claim 10, wherein the silica powder is at least one selected from the group consisting of polymethylsilsesquioxane (PMSQ), vinyl dimethicone/methicone silsesquioxane crosspolymer, silica, and silica silylate.

16. The core stick-type oil dispersion solid cosmetic composition of claim 10, wherein the silica powder is contained in a content of 1.0 to 10.0 wt % relative to the total cosmetic composition.

17. The core stick-type oil dispersion solid cosmetic composition of claim 10, wherein the wax is at least one selected from the group consisting of a hydrogenated vegetable oil, polyethylene, paraffin, microcrystalline wax, candelilla wax, ceresin, polyethylene, and ozokerite.

18. The core stick-type oil dispersion solid cosmetic composition of claim 10, wherein the wax is contained in a content of 1.0 to 10.0 wt % relative to the total cosmetic composition.

19. The core stick-type oil dispersion solid cosmetic composition of claim 10, wherein the surfactant is at least one selected from the group consisting of sorbitan isostearate, polyglyceryl-4 isostearate, sorbitan oleate, polyglyceryl-2 triisostearate, polyglyceryl-2 diisostearate, and polyglyceryl-10 laurate.

20. (canceled)

21. The core stick-type oil dispersion solid cosmetic composition of claim 10, wherein the thickener is at least one selected from the group consisting of diisostearyl malate, VP/hexadecene copolymer, and silica dimethyl silylate.

22. (canceled)
